(19) United States
(12) Patent Application Publication
(10) Pub. No.: US 2012/0165299 A1
(43) Pub. Date: Jun. 28, 2012

(54) COMBINATION MEDICAMENT

(75) Inventor: Anant Sharma, Bedford (GB)

(73) Assignee: Sunil SHAH, Birmingham (GB)

(21) Appl. No.: 13/394,171

(22) PCT Filed: Sep. 3, 2010
(86) PCT No.: PCT/GB2010/001668

§ 371 Date: Mar. 5, 2012

(30) Foreign Application Priority Data
Sep. 3, 2009 (GB) 0915319.8

(51) Int. Cl. A61K 31/573; A61P 37/08; A61K 31/616; A61K 31/57; A61P 29/00; A61P 27/02

(52) U.S. Cl. 514/164; 514/171

(57) ABSTRACT

A medicament for treating inflammation of human or animal body tissues contains a steroid, typically a corticosteroid, in combination with a non-steroidal anti-inflammatory drug (NSAID), typically a cyclooxygenase (COX-1 or COX-2) inhibitor. The medicament is of a particular effectiveness in treating inflammation of and around the eyes, whether caused by infection, irritation or surgery. Further medicaments combine an NSAID with antihistamines or with mast cell stabilisers. The medicaments are particularly effective in topically-applicable form, although other forms of application should also show benefits.

The present invention relates to a combination medicament for treatment of tissue inflammation, including that associated with irritation, allergy, asthma or tissue damage. More particularly but not exclusively, it relates to combination medicament compositions having steroidal active components and offering improved performance over such steroid medicaments alone, especially in the treatment of eye conditions.

Steroids, and corticosteroids in particular, are effective anti-inflammatory drugs, which may be used to treat a wide range of conditions. However, steroids have distinct drawbacks. Many patients have a low tolerance of some or all steroids. For all patients, the risk of adverse side-effects means that it is desirable to administer as low a dose of a steroid as possible, but this must be balanced against the need for efficacy. Similar considerations apply for many other pharmaceuticals, but steroids are of particular concern in this respect.

Steroid preparations are known to be effective in ophthalmic applications, for example in the form of eye-drops. These have been used to treat allergic eye diseases, to alleviate pain and swelling following cataract surgery or refractive surgery, and to treat cystoid macular oedema, amongst other conditions of the eye. Because of the risks of side effects, however, attempts have been made to substitute other pharmaceuticals, such as those generally referred to as non-steroidal anti-inflammatory drugs (NSAIDs). These usually have fewer side-effects. However, as a general rule, NSAIDs are found to be less effective than steroids.

Other drugs used to deal with allergic conditions of the eyes include anti-histamines and mast cell stabilisers, such as sodium cromoglycate. Again, there tends to be a trade-off between effectiveness as a treatment and the risk of side effects (many anti-histamines are liable to cause drowsiness, for example).

While the present invention is primarily concerned with the treatment of ophthalmic conditions, including the after-effects of ophthalmic surgery, the same or similar pharmaceuticals are used to treat a range of other conditions. Considerations similar to those for ophthalmic conditions frequently apply. For example, rhinitis and asthmatic conditions, whether or not of allergic origin, have been treated with analogous drugs to those referred to above but side-effects are again of concern. It is thus believed that medicaments intended originally for ophthalmic use may also be of use in such other applications.

It is hence an object of the present invention to provide medicaments for the treatment of ophthalmic inflammation and analogous conditions that provide a superior balance between effectiveness and adverse effects, compared to existing medicaments.

According to a first aspect of the present invention, there is provided a medicament for the treatment of inflammation of body tissues comprising a pharmaceutically-active steroid combined with a non-steroidal anti-inflammatory drug (NSAID).

Preferably, said steroid comprises a corticosteroid.

Advantageously, said steroid comprises a glucocorticoid steroid.

Said steroid may comprise dexamethasone, fluoromethalone, prednisolone and/or betamethasone.

Preferably, said NSAID comprises a cyclooxygenase (COX) inhibitor.

Said cyclooxygenase inhibitor may comprise a COX-1 inhibitor and/or a COX-2 inhibitor.

Said NSAID may comprise a leukotriene inhibitor, such as montelukast or zileuton.

Said NSAID may comprise ketorolac, diclofenac, indomethacin, ibuprofen and/or aspirin (acetylsalicylic acid).

The medicament may additionally comprise a mast cell stabiliser.

Said mast cell stabiliser may comprise a cromone, optionally sodium cromoglycate.

The medicament may additionally comprise an anti-histamine.

Said anti-histamine may comprise diphenylhydramine, loratadine, desloratadine, meclizine and/or chlorphenamine.

According to a second aspect of the present invention, there is provided a medicament for the treatment of inflammation of body tissues, comprising an anti-histamine together with an NSAID.

Said anti-histamine may comprise diphenylhydramine, loratadine, desloratadine, meclizine and/or chlorphenamine.

Preferably, said NSAID comprises a cyclooxygenase (COX) inhibitor.

Said cyclooxygenase inhibitor may comprise a COX-1 inhibitor and/or a COX-2 inhibitor.

Said NSAID may comprise a leukotriene inhibitor, such as montelukast or zileuton.

Said NSAID may comprise ketorolac, diclofenac, indomethacin, ibuprofen and/or aspirin (acetylsalicylic acid).

According to a third aspect of the present invention, there is provided a medicament for the treatment of inflammation of body tissues, comprising a steroid together with an anti-histamine.

Preferably, said steroid comprises a corticosteroid.

Advantageously, said steroid comprises a glucocorticoid steroid.

Said steroid may comprise dexamethasone, fluoromethalone, prednisolone and/or betamethasone.

Said anti-histamine may comprise diphenylhydramine, loratadine, desloratadine, meclizine and/or chlorphenamine.

According to a fourth aspect of the present invention, there is provided a medicament for the treatment of inflammation of body tissues, comprising a steroid together with a mast cell stabiliser.

Preferably, said steroid comprises a corticosteroid.

Advantageously, said steroid comprises a glucocorticoid steroid.

Said steroid may comprise dexamethasone, fluoromethalone, prednisolone and/or betamethasone.

Said mast cell stabiliser may comprise a cromone, optionally sodium cromoglycate.

According to a fifth aspect of the present invention, there is provided a medicament for the treatment of inflammation of body tissues, comprising an anti-histamine together with a mast cell stabiliser.

Said anti-histamine may comprise diphenylhydramine, loratadine, desloratadine, meclizine and/or chlorphenamine.

Said mast cell stabiliser may comprise a cromone, optionally sodium cromoglycate.

According to a sixth aspect of the present invention, there is provided a medicament for the treatment of inflammation of body tissues, comprising a mast cell stabiliser together with an NSAID.

Said mast cell stabiliser may comprise a cromone, optionally sodium cromoglycate.

Preferably, said NSAID comprises a cyclooxygenase (COX) inhibitor.

Said cyclooxygenase inhibitor may comprise a COX-1 inhibitor and/or a COX-2 inhibitor.

Said NSAID may comprise a leukotriene inhibitor, such as montelukast or zileuton.

Said NSAID may comprise ketorolac, diclofenac, indomethacin, ibuprofen and/or aspirin (acetylsalicylic acid).

According to a seventh aspect of the present invention, there is provided a use of a combination of any two, any three or all of a steroid, an NSAID, an anti-histamine and a mast cell stabiliser in the manufacture of a medicament for the treatment of inflammation of body tissues.

In any one of the first to seventh aspects above, the medicament may be adapted for topical administration.

The medicament may comprise a fluid composition administrable to a patient's eye or its immediate surroundings, such as eye-drops.

The medicament may comprise a cream or ointment.

Alternatively, the medicament may be adapted for parenteral administration.

Said parenteral administration may comprise injection or infusion.

The medicament may be adapted for administration by inhalation.

The medicament may be adapted for oral administration.

The medicament may then comprise a solid preparation such as tablet or capsule means.

Alternatively, the medicament may then comprise a liquid preparation.

The medicament may be adapted for sublingual administration.

The medicament may be adapted for administration between an eyelid and the eye.

The medicament may be adapted for intracameral administration, into a chamber of the eye.

The medicament may be adapted for transdermal administration, for example comprising transdermal patch means.

The medicament may comprise a slow-release composition.

The medicament may be adapted for administration by means of a slow-release device.

According to an eighth aspect of the present invention, there is provided a method of treatment of inflammation of body tissues, comprising the administration of a medicament as described in any one of the first to sixth aspects above.

Said inflammation may comprise inflammation of ocular, intra-ocular or peri-ocular tissues.

Said inflammation may be a consequence of surgical treatment, particularly of the eye and/or its surroundings.

Said inflammation may be a consequence of infection.

Said inflammation may be a consequence of an allergic reaction.

Embodiments of the present invention will now be more particularly described, by way of example.

In a first example, a patient has a severe reaction to an allergen, resulting in inflammation in and around the eyes, and considerable discomfort (this condition may be known as “itchy eye”). The inflammation was not significantly reduced by administration of a mild anti-inflammatory, such as a Non-Steroidal Anti-Inflammatory Drug (NSAID). It was believed that the inflammation would be susceptible to conventional treatment with steroid eye-drops, but the possible side-effects of the levels of steroid usually required were a matter of concern.

An eye-drop formulation was made up containing both the glucocorticoid steroid dexamethasone and the NSAID diclofenac, a remainder of the eye-drop formulation being of conventional form. This eye-drop formulation was administered according to a dosage regime similar to that conventionally employed for treatment with eye-drops containing a steroid alone. Significant alleviation of the inflammation and discomfort was detected at net steroid levels significantly lower than those conventionally employed in such a treatment. Considering the relatively low efficacy of NSAIDs alone for this purpose, the presence of a synergistic effect between the steroid and the NSAID was unexpected.

Similar results were achieved from the local application of a cream or ointment containing both dexamethasone and diclofenac.

In a second example, a patient presents with the ophthalmic condition cystoid macular oedema. A conventional treatment would comprise administration of a course of steroid eye-drops, but there is concern (as is often the case) that the required levels of steroid might lead to undesirable interactions with medication being taken by the patient for another, unconnected condition.

The eye-drop formulation described above, comprising dexamethasone and diclofenac, should be administered at net steroid levels significantly below those normally required for successful treatment with dexamethasone alone. Nevertheless, on the basis of the results achievable in the first example, above, a major reduction in oedema would be expected.

In a third example, a cataract is removed from a patient's eye by standard surgical techniques. Conventionally, post-operative inflammation would be managed using eye-drops containing the steroid fluoromethasone.

An eye-drop formulation would instead be prepared comprising fluoromethasone (at approximately half the conventional dosage) and the NSAID ketorolac (at levels that would not be expected to be efficacious when used alone), plus conventional excipients. Administration of this formulation according to a conventional dosage regime would be expected to lead to a rapid reduction in post-operative inflammation but without appreciable side-effects from the fluoromethasone.

In a fourth example, a patient undergoes laser refractive surgery, and eye-drops based on fluoromethasone would normally have been prescribed to control post-operative inflammation. However, the above formulation, based on fluoromethasone combined with ketorolac, would be used instead, and should provide effective treatment at significantly lower net levels of steroid administration.

In a fifth example, a patient presents with severe acute rhinitis and eye inflammation as a result of an allergic reaction. Normally, a steroidal ointment would be administered to the eyes, and a steroidal nasal spray would be used to treat rhinitis. However, owing to a pre-existing history of glaucoma, it is in this case desired to minimise levels of steroids, which might raise intra-ocular pressure.

Therefore, an eye-drop formulation and a nasal spray should be prepared, each of largely conventional form. However, each would comprise prednisolone at half the normal level, combined with diclofenac. Administration of these medicaments should alleviate a majority of the patient's symptoms, and no significant increase in intra-ocular pressure would be expected.

Although the above examples are of tests on human beings, there is no reason to believe that similar results would not be achieved in veterinary use.

It is hypothesized that the synergistic effects noted with combinations containing an NSAID may result from the NSAID mediating the passage of the other active component or components through the surface or outer layers of the respective body tissues. Thus, the onset of effect of the active component(s) may be speeded and/or their effective concentration reaching the target tissue to be treated may be increased.

Note: it appears to be a matter of debate whether Paracetamol (also known as acetaminophen) qualifies as an NSAID. For the purposes of this application, the term NSAID may be understood to include Paracetamol/acetaminophen, unless it is clear from the context that this is not the case.

What is claimed is:

1. A medicament for the treatment of inflammation of body tissues comprising a pharmaceutically-active steroid and a non-steroidal anti-inflammatory drug (NSAID).

2. A medicament as claimed in claim 1, wherein said steroid comprises a corticosteroid.

3. A medicament as claimed in claim 1, wherein said steroid comprises a glucocorticoid steroid.

4. A medicament as claimed in claim 1, wherein said steroid is selected from the group consisting of dexamethasone, fiuoromethalone, prednisolone, betamethasone, and mixtures thereof.

5. A medicament as claimed in claim 1, wherein said NSAID comprises a cyclooxygenase (COX) inhibitor.

6. A medicament as claimed in claim 5, wherein said cyclooxygenase inhibitor is selected from the group consisting of a COX-1 inhibitor, a COX-2 inhibitor, and mixtures thereof.

7. A medicament as claimed in claim 5, wherein said NSAID comprises a leukotriene inhibitor, selected from montelukast and zileuton.

8. A medicament as claimed in claim 5, wherein said NSAID is selected from the group consisting of ketorolac, diclofenac, indomethacin, iso-butyl-propanoic-phenolic acid, acetylsalicylic acid, and mixtures thereof.

9. A medicament as claimed in claim 1, further comprising a mast cell stabiliser.

10. A medicament as claimed in claim 1, further comprising an anti-histamine selected from the group consisting of diphenylhydramine, loratadine, desloratadine, meclizine, chlorphenamine, and mixtures thereof. cm 11-20. (canceled)

21. A medicament comprising:
a first agent; and
a second agent; wherein the first and second agents are selected from the group consisting of a pharmaceutically-active steroid, an NSAID, an anti-histamine, a mast cell stabiliser, and mixtures thereof, adapted for the treatment of inflammation of body tissues.

22. A medicament as claimed in claim 1, adapted for topical administration or transdermal administration.

23. A medicament as claimed in claim 1, comprising a fluid composition administrable to a patient's eye or its immediate surrounding.

24. (canceled)

25. A medicament as claimed in claim 1, comprising a slow-release composition.

26. A method of treatment of inflammation of body tissues of a human or an animal, comprising the administration of a medicament as claimed in claim 1.

27. A method of treatment as claimed in claim 26, wherein said inflammation comprises inflammation of ocular, intra-ocular or peri-ocular tissues.

28. A method of treatment as claimed in claim 26, wherein said inflammation is a consequence of a surgical treatment of the eye and/or its surroundings, an infection, or an allergic reaction.

29. (canceled)

30. (canceled)

31. A medicament as claimed in claim 9, wherein the mast cell stabilizer is a cromone.

32. A medicament as claimed in claim 31, wherein the cromone is sodium cromoglycate.

33. A medicament comprising:
a pharmaceutically-active steroid;
a non-steroidal anti-inflammatory drug;
a mast cell stabilizer; and
anti-histamine.
